(19) United States
(12) Patent Application Publication
(10) Pub. No.: US 2009/0117158 A1
(43) Pub. Date: May 7, 2009

(54) Transdermal sustained release drug delivery

(76) Inventors: Mahmoud Ameri, Fremont, CA (US); Yuh-Fun Maa, Millbrae, CA (US)

Correspondence Address: Edwards Angell Palmer & Dodge LLP, P.O. Box 55874, Boston, MA, 02205, US

(21) Appl. No.: 12/288,760

(22) Filed: Oct. 23, 2008

Related U.S. Application Data
(60) Provisional application No. 60/982,127, filed on Oct. 23, 2007.

(51) Int. Cl. A61K 9/00; A61K 38/20; A61K 38/16; B05D 3/00

(52) U.S. Cl. 424/400; 424/85.2; 514/12; 427/2.31

(57) ABSTRACT

Provided herein are microprojections and microprojection arrays wherein a microprojection is coated with at least two layers. One layer comprises a biologically active agent, for example, a PTH agent and optionally other excipients. Another layer, which is generally, initially devoid of active agent comprises a polymer or a mix of polymers to provide controlled release, for example sustained release, of the biologically active agent contained in the first layer. Microprojections coated with multiple layers, some layers containing a biologically active agent and other layers containing a polymer for controlled release are also contemplated herein.

### FIELD OF THE PRESENT INVENTION

The present invention relates generally to biologically active agent compositions and methods for formulating and delivering such compositions. More particularly, the present invention relates to transdermal sustained release drug delivery active agent compositions to oxygen and water.

### BACKGROUND OF THE INVENTION

A great number and variety of biologically active agents are known in the art to have therapeutic benefits when delivered appropriately to a patient having a condition upon which such biologically active agents can exert a beneficial effect. These biologically active agents comprise several broad classes, including, but not limited to peptides or proteins, such as hormones, proteins, antigens, repressors/activators, enzymes, and immunoglulins, among others. Therapeutic applications include treatment of cancer, hypercalcemia, Paget's disease, osteoporosis, diabetes, cardiac conditions, including congestive heart failure, sleep disorders, Chronic Obstructive Pulmonary Disease (COPD) and anabolic conditions, to name a few.

Active agents (or drugs) are most conventionally administered either orally or by injection. Unfortunately, many active agents are completely ineffective or have radically reduced efficacy when orally administered, since they either are not absorbed or are adversely affected before entering the bloodstream and thus do not possess the desired activity. On the other hand, the direct injection of the agent intravenously or subcutaneously, while assuring no modification of the agent during administration, is a difficult, inconvenient, painful and uncomfortable procedure that sometimes results in poor patient compliance.

Hence, in principle, transdermal delivery provides for a method of administering active agents that would otherwise need to be delivered via hypodermic injection or intravenous infusion. The word “transdermal”, as used herein, is generic term that refers to delivery of an active agent (e.g., a therapeutic agent, such as a drug or an immunologically active agent, such as a vaccine) through the skin to the local tissue or systemic circulatory system without substantial cutting or penetration of the skin, such as cutting with a surgical knife or piercing the skin with a hypodermic needle. Transdermal agent delivery includes delivery via passive diffusion as well as delivery based upon external energy sources, such as electricity (e.g., iontophoresis) and ultrasound (e.g., phonophoresis).

Passive transdermal agent delivery systems, which are more common, typically include a drug reservoir that contains a high concentration of an active agent. The reservoir is adapted to contact the skin, which enables the agent to diffuse through the skin and into the body tissues or bloodstream of a patient.

As is well known in the art, the transdermal drug flux is dependent upon the condition of the skin, the size and physical/chemical properties of the drug molecule, and the concentration gradient across the skin. Because of the low permeability of the skin to many drugs, transdermal delivery has had limited applications. This low permeability is attributed primarily to the stratum corneum, the outermost skin layer which consists of flat, dead cells filled with keratin fibers (i.e., keratinocytes) surrounded by lipid bilayers. This highly-ordered structure of the lipid bilayers confers a relatively impermeable character to the stratum corneum.

One common method of increasing the passive transdermal diffusional agent flux involves pre-treating the skin with, or co-delivering with the agent, a skin permeation enhancer. A permeation enhancer, when applied to a body surface through which the agent is delivered, enhances the flux of the agent therethrough. However, the efficacy of these methods in enhancing transdermal protein flux has been limited, at least for the larger proteins, due to their size.

There also have been many techniques and devices developed to mechanically penetrate or disrupt the outermost skin layers thereby creating pathways into the skin in order to enhance the amount of agent being transdermally delivered. Illustrative is the drug delivery device disclosed in U.S. Pat. No. 3,964,482.

Other systems and apparatus that employ tiny skin piercing elements to enhance transdermal agent delivery are disclosed in U.S. Pat. Nos. 5,879,326, 3,814,097, 5,250,023, 3,964,482, Reissue No. 25,637, and PCT Publication Nos. WO 96/37155, WO 96/37256, WO 96/17648, WO 97/03718, WO 98/11937, WO 98/00193, WO 97/48440, WO 97/48441, WO 97/48442, WO 98/00193, WO 99/64580, WO 98/28037, WO 98/29298, and WO 98/29365; all incorporated herein by reference in their entirety.

The disclosed systems and apparatus employ piercing elements of various shapes and sizes to pierce the outermost layer (i.e., the stratum corneum) of the skin. The piercing elements disclosed in these references generally extend perpendicularly from a thin, flat member, such as a pad or sheet. The piercing elements in some of these devices are extremely small, some having a microprojection length of only about 25-400 microns and a microprojection thickness of only about 5-50 microns. These tiny piercing/cutting elements make correspondingly small microslits/microcuts in the stratum corneum for enhancing transdermal agent delivery therethrough.

The disclosed systems further typically include a reservoir for holding the agent and also a delivery system to transfer the agent from the reservoir through the stratum corneum, such as by hollow tines of the device itself. One example of such a device is disclosed in WO 93/17754, which has a liquid agent reservoir. The reservoir must, however, be pressurized to force the liquid agent through the tiny tubular elements and into the skin. Disadvantages of such devices include the added complication and expense for adding a pressurizable liquid reservoir and complications due to the presence of a pressure-driven delivery system.

As disclosed in U.S. patent application Ser. No. 10/045,842, which is fully incorporated by reference herein, it is possible to have the active agent that is to be delivered coated on the microprojections instead of contained in a physical reservoir. This eliminates the necessity of a separate physical reservoir and developing an agent formulation or composition specifically for the reservoir.

It is another object of the present invention to provide a transdermal agent delivery apparatus that can be used to prevent or treat osteopenia in order to prevent or minimize the onset of osteoporosis, osteoporotic fractures, and other osteoporosis-related disorders.

### SUMMARY OF THE INVENTION

In accordance with the above objects and those that will be mentioned and will become apparent below, the apparatus and method for transdermally delivering a biologically active agent, for example a PTH based agent in accordance with this invention generally comprises a delivery system having a microprojection member (or system) that includes a plurality of microprojections (or array thereof) that are adapted to pierce through the stratum corneum into the underlying epidermis layer, or epidermis and dermis layers.

As will be apparent through the foregoing specification; provided herein are microprojections and microprojection arrays wherein the microprojection is coated with at least two layers. One layer comprises a biologically active agent, for example, a PTH agent and optionally other excipients. Another layer, which is generally, initially devoid of active agent comprises a polymer or a mix of polymers to provide controlled release, for example sustained release, of the biologically active agent contained in the first layer. Microprojections coated with multiple layers, some layers containing a biologically active agent and other layers containing a polymer for controlled release are contemplated herein.

Drug containing layers are formed based on a drug coating formulation. Accordingly, formulations employed herein in the formation of a coating layer containing a biologically active agent will be referred to as drug coating formulations.

Likewise, formulations used in the formation of a polymer layer that controls the release of the biologically active agent are referred to a controlled release coating formulations.

One embodiment provides a transdermal delivery device for delivering a biologically active agent comprising at least one stratum corneum-piercing microprojection, wherein said microprojection has a first coating comprising said biologically active agent and a second coating comprising a polymer, wherein the polymer coating allows controlled release of said biological agent after the transdermal delivery device is applied to the skin of a subject.

Another embodiment provides a transdermal delivery device for delivering a biologically active agent comprising at least one stratum corneum-piercing microprojection, wherein said microprojection has a plurality of coating layers; wherein at least one coating layer comprises said biologically active agent and at least one coating layer comprises a polymer, wherein the polymer coating allows controlled release of said biological agent after the transdermal delivery device is applied to the skin of a subject.

Yet another embodiment provides a transdermal delivery device wherein coating layers comprising the biologically active agent and coating layers comprising the controlled release polymer are alternately disposed on said microprojection.

Still another embodiment provides a device wherein the polymer is a hydrophilic polymer or a hydrophobic PLGA copolymer.

Still another embodiment provides a device wherein the polymer layer has a thickness selected to provide a predetermined sustained release profile for the biologically active agent.

A further embodiment provides a device wherein the polymer layer has a copolymer molar mass, a copolymer architecture, a water hydration rate, and/or or layer thickness selected to provide a predetermined sustained release profile for the biologically active agent.

Yet another embodiment provides a device wherein the controlled release profile comprises a shorter tmax and a rapid concentration drop off.

Still another embodiment provides a device wherein the controlled release comprises a reduced Cmax and an extended drop-off tail.

In still another embodiment provides a device of wherein the polymer layer encapsulates the biologically active agent and slows down release of the biologically active agent.

In still another embodiment provides a device of wherein the biologically active agent is selected from the group consisting of growth hormone release hormone (GHRH), growth hormone release factor (GHRF), insulin, insultropin, calcitonin, octreotide, endorphin, TRN, NT-36 (chemical name: N-[[(s)-4-oxo-2-azetidinyl] carbonyl]-L-histidyl-L-prolinamide), liprecin, pituitary hormones (e.g., HGH, HMG, desmopressin acetate, etc), follicle luteoids, aANF, growth factors such as growth factor releasing factor (GFRF), bMSH, GH, somatostatin, bradykinin, somatotropin, platelet-derived growth factor releasing factor, asparaginase, bleomycin sulfate, chymopapain, cholecystokinin, chorionic gonadotropin, erythropoietin, epoprostenol (platelet aggregation inhibitor), gluagon, HCG, hirulog, hyaluronidase, interferon alpha, interferon beta, interferon gamma, interleukins, interleukin-10 (IL-10), erythropoietin (EPO), granulocyte macrophage colony stimulating factor (GM-CSF), granulocyte colony stimulating factor (G-CSF), glucagon, leutinizing hormone releasing hormone (LHRH), LHRH analogs (such as goserelin, leuprolide, buserelin, triptorelin, gonadorelin, and napfarelin, menotropins (urofollitropin (FSH) and LH)), oxytocin, streptokinase, tissue plasminogen activator, urokinase, vasopressin, deamino [Val4, D-Arg8] arginine vasopressin, desmopressin, corticotropin (ACTH), ACTH analogs such as ACTH (1-24), ANP, ANP clearance inhibitors, angiotensin II antagonists, antidiuretic hormone agonists, bradykinn antagonists, ceredase, CSI's, calcitonin gene related peptide (CGRP), enkephalins, FAB fragments, IgE peptide suppressors, IGF-1, neurotrophic factors, colony stimulating factors, parathyroid hormone and agonists, parathyroid hormone antagonists, parathyroid hormone (PTH), PTH analogs such as PTH (1-34), prostaglandin antagonists, pentigetide, protein C, protein S, renin inhibitors, thymosin alpha-1, thrombolytics, TNF, vasopressin antagonists analogs, alpha-1 antitrypsin (recombinant), and TGF-beta.

Still another embodiment provides a device wherein the biologically active agent comprises hPTH.

In still another embodiment provides a device wherein the biologically active agent comprises hPTH(1-34) and analogs.

In one embodiment of the invention, the microprojection member has a n 2 microprojection density of at least approximately 10 microprojections/cm², more preferably, in the range of at least approximately 200-2000 microprojections/cm².

In one embodiment, the microprojection member is constructed out of stainless steel, titanium, nickel titanium alloys, or similar biocompatible materials.

In another embodiment, the microprojection member is constructed out of a non-conductive material, such as a polymeric material. Alternatively, the microprojection member can be coated with a non-conductive material, such as Parylene®, or a hydrophobic material, such as Teflon®, silicon or other low energy material.

The drug coating formulations applied to the microprojection member to form solid biocompatible coatings can comprise aqueous and non-aqueous formulations. Preferably, the drug coating formulations include at least one biologically active agent, for example a PTH based agent, which can be dissolved within a biocompatible carrier or suspended within the carrier.

In a preferred embodiment, the PTH-based agent is selected from the group consisting of hPTH(1-34), hPTH salts and analogs, teriparatide and related peptides. Throughout this application, the terms “PTH-based agent” and “hPTH(1-34) agent” include, without limitation, recombinant hPTH(11-34), synthetic hPTH(11-34), PTH(11-34), teriparatide, hPTH(1-34) salts, simple derivatives of hPTH(1-34), such as hPTH(1-34) amide, and closely related molecules, such as hPTH(1-33) or hPTH(1-31) amide, or any other closely related osteogenic peptide. Synthetic hPTH(1-34) is the most preferred PTH agent.

Examples of pharmaceutically acceptable hPTH salts include, without limitation, acetate, propionate, butyrate, pentanoate, hexanoate, heptanoate, levulinate, chloride, bromide, citrate, succinate, maleate, glycolate, gluconate, glucuronate, 3-hydroxyisobutyrate, tricarballylicate, malonate, adipate, citraconate, glutarate, itaconate, mesaconate, citramalate, dimethylolpropinate, tiglicate, glycerate, methacrylate, isocrotonate, \tilde{□}hydroxibutyrate, crotonate, angelate, hydracrylate, ascorbate, aspartate, glutamate, 2-hydroxyisobutyrate, lactate, malate, pyruvate, fumarate, tartarate, nitrate, phosphate, benzene, sulfonate, methane sulfonate, sulfate and sulfonate.

Preferably, the biologically active agent, for example a PTH based agent is present in the drug coating formulation at a concentration in the range of approximately 1-30 wt. %.

More preferably, the amount of biologically active agent, for example a PTH based agent contained in the solid biocompatible coating (i.e., microprojection member or product) is in the range of approximately 1 μg-1000 μg, even more preferably, in the range of approximately 10-100 μg.

Also preferably, the pH of the drug coating formulation is below approximately pH 6. More preferably, the drug coating formulation has a pH in the range of approximately pH 2-pH 6. Even more preferably, the drug coating formulation has a pH in the range of approximately pH 3-pH 6.

In one embodiment of the invention, the drug coating formulation includes at least one antioxidant, which can comprise sequestering agents, such sodium citrate, citric acid, EDTA (ethylene-dinitrilo-tetraacetic acid) or free radical scavengers, such as ascorbic acid, methionine, sodium ascorbate and the like. Presently preferred antioxidants comprise EDTA and methionine.

In the noted embodiments of the invention, the concentration of the antioxidant is preferably in the range of approximately 0.01-20 wt. % of the drug coating formulation. More preferably, the concentration of the antioxidant is in the range of approximately 0.03-10 wt. % of the drug coating formulation.

In one embodiment of the invention, the drug coating formulation includes at least one surfactant, which can be zwitterionic, amphoteric, cationic, anionic, or nonionic, including, without limitation, sodium lauroamphoacetate, sodium dodecyl sulfate (SDS), cetylpyridinium chloride (CPC), dodecyltrimethyl ammonium chloride (TMAC), benzalkonium, chloride, polysorbates such as Tween 20 and Tween 80, other sorbitan derivatives, such as sorbitan lauratealkoxylated alcohols, such as laureth-4 and polyoxyethylene castor oil derivatives, such as Cremophor EL®.

In the noted embodiments of the invention, the concentration of the surfactant is preferably in the range of approximately 0.01-20 wt. % of the drug coating formulation. Preferably, the concentration of the surfactant is in the range of approximately 0.05-1 wt. % of the drug coating formulation.

In a further embodiment of the invention, the drug coating formulation includes at least one polymeric material or polymer that has amphiphilic properties, which can comprise, without limitation, cellulose derivatives, such as hydroxyethylcellulose (HEC), hydroxypropylmethylcellulose (HPMC), hydroxypropycellulose (HPC), methylcellulose (MC), hydroxyethylmethylcellulose (HEMC), or ethylhydroxy-ethylcellulose (EHEC), as well as pluronics.

In one embodiment of the invention, the concentration of the polymer presenting amphiphilic properties in the drug coating formulation is preferably in the range of approximately 0.01-20 wt. %, more preferably, in the range of approximately 0.03-10 wt. % of the drug coating formulation.

In another embodiment, the drug coating formulation includes a hydrophilic polymer selected from the following group: hydroxyethyl starch, carboxymethyl cellulose and salts of, dextran, poly(vinyl alcohol), poly(ethylene oxide), poly(2-hydroxyethyl-methacrylate), poly(n-vinyl pyrolidone), polyethylene glycol and mixtures thereof, and like polymers.

In a preferred embodiment, the concentration of the hydrophilic polymer in the drug coating formulation is in the range of approximately 1-30 wt. %, more preferably, in the range of approximately 1-20 wt. % of the drug coating formulation.

In another embodiment of the invention, the drug coating formulation includes a biocompatible carrier, which can comprise, without limitation, human albumin, bioengineered human albumin, polyglutamic acid, polyaspartic acid, polyhistidine, pentosan polysulfate, polyamino acids, sucrose, trehalose, melezitose, raffinose and stachyose.

Preferably, the concentration of the biocompatible carrier in the drug coating formulation is in the range of approximately 2-70 wt. %, more preferably, in the range of approximately 5-50 wt. % of the drug coating formulation.

In another embodiment, the drug coating formulation includes a stabilizing agent, which can comprise, without limitation, a non-reducing sugar, a polysaccharide or a reducing sugar.

Suitable non-reducing sugars for use in the methods and compositions of the invention include, for example, sucrose, trehalose, stachyose, or raffinose.

Suitable polysaccharides for use in the methods and compositions of the invention include, for example, dextran, soluble starch, dextrin, and insulin.

Suitable reducing sugars for use in the methods and compositions of the invention include, for example, monosaccharides such as, for example, apiose, arabinose, lyxose, ribose, xylose, digitoxose, fucose, quercitol, quinovose, rhamnose, allose, altrose, fructose, galactose, glucose, gulose, hamamelose, idose, mannose, tagatose, and the like; and disaccharides such as, for example, primeverose, vicianose, rutinose, scillabiose, cellobiose, gentiobiose, lactose, lactulose, maltose, melibiose, sophorose, and turanose and the like.

Preferably, the concentration of the stabilizing agent in the drug coating formulation is at a ratio of approximately 0.1-2.0:1 with respect to the biologically active agent, for example a PTH based agent, more preferably, approximately 0.25-1.0:1 with respect to the biologically active agent, for example a PTH based agent.

In another embodiment, the drug coating formulation includes a vasoconstrictor, which can comprise, without limitation, amidephrine, cafaminol, cyclopentamine, deoxyepinephrine, epinephrine, felypressin, indanazoline, metizoline, midodrine, naphazoline, nordefrin, octodrine, ornipressin, oxymethazoline, phenylephrine, phenylethanolamine, phenylpropanolamine, propylhexedrine, pseudoephedrine, tetrahydrozoline, tramazoline, tuaminoheptane, tymazoline, vasopressin, xylometazoline and the mixtures thereof. The most preferred vasoconstrictors include epinephrine, naphazoline, tetrahydrozoline indanazoline, metizoline, tramazoline, tymazoline, oxymetazoline and xylometazoline.

The concentration of the vasoconstrictor, if employed, is preferably in the range of approximately 0.1 wt. % to 10 wt. % of the drug coating formulation.

In another embodiment of the invention, the drug coating formulation includes at least one “pathway patency modulator”, which can comprise, without limitation, osmotic agents (e.g., sodium chloride), zwitterionic compounds (e.g., amino acids), and anti-inflammatory agents, such as betamethasone 21-phosphate disodium salt, triamcinolone acetonide 21-disodium phosphate, hydrocortamate hydrochloride, hydrocortisone 21-phosphate disodium salt, methylprednisolone 21-phosphate disodium salt, methylprednisolone 21-succinate sodium salt, paramethasone disodium phosphate and prednisolone 21-succinate sodium salt, and anticoagulants, such as citric acid, citrate salts (e.g., sodium citrate), dextrin sulfate sodium, aspirin and EDTA.

In yet another embodiment of the invention, the drug coating formulation includes a solubilising/complexing agent, which can comprise Alpha-Cyclodextrin, Beta-Cyclodextrin, Gamma-Cyclodextrin, glucosyl-alpha-Cyclodextrin, maltosyl-alpha-Cyclodextrin, glucosyl-beta-Cyclodextrin, maltosyl-beta-Cyclodextrin, hydroxypropyl beta-Cyclodextrin, 2-hydroxypropyl-beta-Cyclodextrin, 2-hydroxypropyl-gamma-Cyclodextrin, hydroxyethyl-beta-Cyclodextrin, methyl-beta-Cyclodextrin, sulfobutylether-alpha-Cyclodextrin, sulfobutylether-beta-Cyclodextrin, and sulfobutylether-gamma-Cyclodextrin. Most preferred solubilising/complexing agents are beta-Cyclodextrin, hydroxypropyl beta-Cyclodextrin, 2-hydroxypropyl-beta-Cyclodextrin and sulfobutylether7 beta-Cyclodextrin.

The concentration of the solubilising/complexing agent, if employed, is preferably in the range of approximately 1 wt. % to 20 wt. % of the drug coating formulation.

In another embodiment of the invention, the drug coating formulation includes at least one non-aqueous solvent, such as ethanol, isopropanol, methanol, propanol, butanol, propylene glycol, dimethysulfoxide, glycerin, N,N-dimethylformamide and polyethylene glycol 400. Preferably, the non-aqueous solvent is present in the drug coating formulation in the range of approximately 1 wt. % to 50 wt. % of the drug coating formulation.

Preferably, the drug coating formulations have a viscosity less than approximately 500 centipoise and greater than 3 centipoise.

In one embodiment of the invention, the thickness of the biocompatible coating is less than 25 microns, more preferably, less than 10 microns, as measured from the microprojection surface.

In accordance with one embodiment of the invention, the method for delivering a biologically active agent, for example a PTH based agent to a subject comprises (i) providing a microprojection member having a plurality of stratum corneum-piercing microprojections, the microprojection member having a biocompatible coating disposed thereon that includes at least one biologically active agent, for example a PTH based agent, (ii) applying the microprojection member to a skin site on the subject, whereby the microprojections pierce the stratum corneum and deliver the biologically active agent, for example a PTH based agent to the subject.

Preferably, the coated microprojection member is applied to the skin site via an impact applicator.

Also preferably, the coated microprojection member is preferably left on the skin site for a period lasting from 5 seconds to 24 hours. Following the desired wearing time, the microprojection member is removed. In some embodiments, wherein the biologically active agent, for example a PTH based agent is in the range of approximately 1 μg-1000 μg of the biocompatible coating.

In the methods of the invention, transdermal delivery of a biologically active agent, for example a PTH based agent preferably exhibits rapid on-set of biological action. Also preferably, transdermal delivery of a biologically active agent, for example a PTH based agent exhibits sustained biological action for a period of up to 8 hours.

In one embodiment, the transdermally delivered PTH-based agent comprises teriparatide (hPTH (1-34)) and the biocompatible coating comprises a dose of the PTH-based agent in the range of approximately 10-100 μg dose, wherein delivery of the PTH-based agent results in a plasma Cₘₐₓ of at least 50 pg/mL after one application.

The invention also comprises a method of improving the pharmacokinetics of a transdermally delivered biologically active agent, for example a PTH based agent comprising providing a microprojection member having a plurality of stratum corneum-piercing microprojections, the microprojection member having a biocompatible coating disposed thereon that includes at least one biologically active agent, for example a PTH based agent and applying the microprojection member to a skin site on the subject, whereby the microprojections pierce the stratum corneum and deliver the biologically active agent, for example a PTH based agent to the subject so that delivery of the biologically active agent, for example a PTH based agent has improved pharmacokinetics compared to the pharmacokinetics characteristic of subcutaneous delivery.

### BRIEF DESCRIPTION OF THE DRAWINGS

Further features and advantages will become apparent from the following and more particular description of the preferred embodiments of the invention, as illustrated in the accompanying drawings, and in which like referenced characters generally refer to the same parts or elements throughout the views, and in which:

FIG. 1 is a perspective view of a portion of one example of a microprojection member, according to the invention;

FIG. 2 is a perspective view of the microprojection member shown in FIG. 1 having a coating deposited on the microprojections, according to the invention;

FIG. 3 is a side sectional view of a microprojection member having an adhesive backing, according to the invention;

FIG. 4 is a side sectional view of a retainer having a microprojection member disposed therein, according to the invention;

FIG. 5 is a perspective view of the retainer shown in FIG. 4;

FIG. 6 is an exploded perspective view of an applicator and retainer, according to the invention;

FIG. 7 is a blowout of a microprojection with drug layer and polymer layer according to the invention;

FIG. 8 shows a PTH coated array according to the invention;

FIG. 9 shows a microprojection array with PTH and Pluroinic F127 coatings according to the invention;

FIG. 10 shows a microprojection array with PTH and PVP K30 coatings according to the invention; and

FIG. 11 shows a microprojection array with PTH and Dextran 67 KDa coatings according to the invention.

### DETAILED DESCRIPTION OF THE INVENTION

Before describing the present invention in detail, it is to be understood that this invention is not limited to particularly exemplified materials, methods or structures as such may, of course, vary. Thus, although a number of materials and methods similar or equivalent to those described herein can be used in the practice of the present invention, the preferred materials and methods are described herein.

It is also to be understood that the terminology used herein is for the purpose of describing particular embodiments of the invention only and is not intended to be limiting.

Unless defined otherwise, all technical and scientific terms used herein have the same meaning as commonly understood by one having ordinary skill in the art to which the invention pertains.

Further, all publications, patents and patent applications cited herein, whether supra or infra, are hereby incorporated by reference in their entirety.

Finally, as used in this specification and the appended claims, the singular forms “a, “an” and “the” include plural referents unless the content clearly dictates otherwise. Thus, for example, reference to “an active agent” includes two or more such agents; reference to “a microprojection” includes two or more such microprojections and the like.

### DEFINITIONS

The term “transdermal”, as used herein, means the delivery of an agent into and/or through the skin for local or systemic therapy.

The term “transdermal flux”, as used herein, means the rate of transdermal delivery.

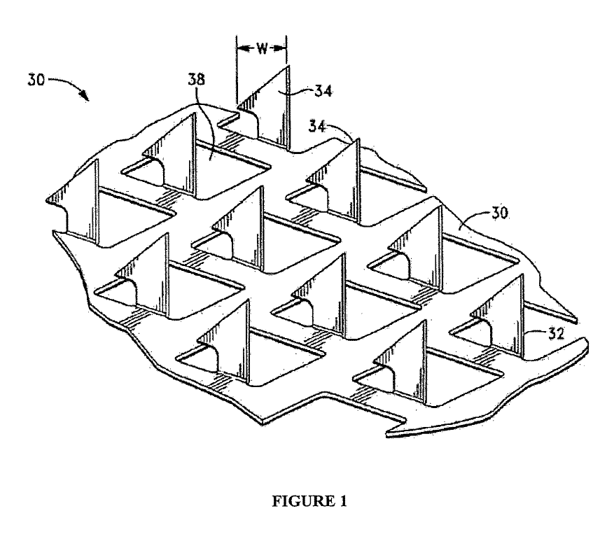

The terms “pulsatile delivery profile” and “pulsatile concentration profile”, as used herein, mean a post administration increase in blood serum concentration of a biologically active agent, for example a PTH based agent from a baseline concentration to a concentration in the range of approximately 50-1000 pg/mL in a period ranging from 1 min. to 4 hr., wherein Cₘₐₓ is achieved, and a decrease in blood serum concentration from Cₘₐₓ to the baseline concentration in a period ranging from 1-8 hrs. after Cₘₐₓ has been achieved. As illustrated in FIG. 1, the noted concentration (or pharmacokinetic) profile typically reflects a rapid rise in blood serum concentration after administration (i.e., first region) and a slightly less rapid decline (i.e., second region) relative to the first region after Cₘₐₓ has been reached, which is generally reflected by a spike in the concentration profile.

Other concentration profiles resulting in a pulsatile delivery comprising a rise in blood concentration of the biologically active agent, for example a PTH based agent to a Cₘₐₓ of 50-1000 pg/mL within a twelve-hour period following administration would also likely result in the desired beneficial effect and, hence, are within the scope of the present invention.

The term “co-delivering”, as used herein, means that a supplemental agent(s) is administered transdermally either before the biologically active agent, for example a PTH based agent is delivered, before and during transdermal flux of the biologically active agent, for example a PTH based agent, during transdermal flux of the biologically active agent, for example a PTH based agent, during and after transdermal flux of the biologically active agent, for example a PTH based agent, and/or after transdermal flux of the PTH-based agent. Additionally, two or more PTH-based agents may be formulated in the coatings and/or formulations, resulting in co-delivery of the PTH-based agents.

The terms “PTH-based agent” and “hPTH(1-34) agent”, as used herein, include, without limitation, hPTH(11-34), hPTH salts, hPTH analogs, teriparatide, closely related peptides and agents having a peptide sequence that functions by the same means as the 34 N-terminal amino acids (the biologically active region) sequence of the 84-amino acid human parathyroid hormone. The terms “PTH-based agent” and “hPTH(1-34) agent” thus include, without limitation, recombinant hPTH(1-34), synthetic hPTH(1-34), PTH(1-34), hPTH(1-34) salts, teriparatide, simple derivatives of hPTH(1-34), such as hPTH(1-34) amide and closely related molecules, such as hPTH(1-33) or hPTH(1-31) amide and closely related osteogenic peptides.

Examples of suitable hPTH salts include, without limitation, acetate, propionate, butyrate, pentanoate, hexanoate, heptanoate, levulinate, chloride, bromide, citrate, succinate, maleate, glycolate, gluconate, glucuronate, 3-hydroxyisobutyrate, tricarballylicate, malonate, adipate, citraconate, glutarate, itaconate, mesaconate, citramalate, dimethylolpropinate, tiglicate, glycerate, methacrylate, isocrotonate, □̅hydroxibutyrate, crotonate, angelate, hydracrylate, ascorbate, aspartate, glutamate, 2-hydroxyisobutyrate, lactate, malate, pyruvate, fumarate, tartarate, nitrate, phosphate, benzene, sulfonate, methane sulfonate, sulfate and sulfonate.

The noted PTH-based agents can also be in various forms, such as free bases, acids, charged or uncharged molecules, components of molecular complexes or nonirritating, pharmacologically acceptable salts.

It is to be understood that more than one PTH-based agent can be incorporated into the agent source, reservoirs, and/or coatings of this invention, and that the use of the term “PTH-based agent” in no way excludes the use of two or more such agents.

The term “microprojections”, as used herein, refers to piercing elements which are adapted to pierce or cut through the stratum corneum into the underlying epidermis layer, or epidermis and dermis layers, of the skin of a living animal, particularly a mammal and more particularly a human.

In one embodiment of the invention, the piercing elements have a projection length less than 1000 microns. In a further embodiment, the piercing elements have a projection length of less than 500 microns, more preferably, less than 250 microns. The microprojections further have a width (designated “W” in FIG. 1) in the range of approximately 25-500 microns and a thickness in the range of approximately 10-100 microns. The microprojections may be formed in different shapes, such as needles, blades, pins, punches, and combinations thereof.

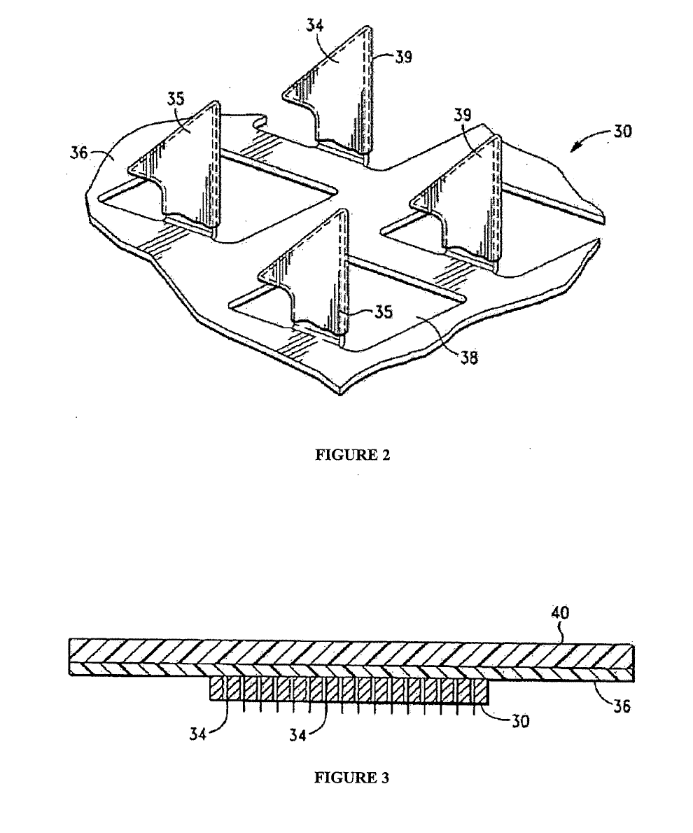

The term “microprojection member”, as used herein, generally connotes a microprojection array comprising a plurality of microprojections arranged in an array for piercing the stratum corneum. The microprojection member can be formed by etching or punching a plurality of microprojections from a thin sheet and folding or bending the microprojections out of the plane of the sheet to form a configuration, such as that shown in FIG. 2. The microprojection member can also be formed in other known manners, such as by forming one or more strips having microprojections along an edge of each of the strip(s) as disclosed in U.S. Pat. No. 6,050,988, which is hereby incorporated by reference in its entirety.

The term “drug coating formulation”, as used herein, is meant to mean and include a freely flowing composition or mixture that is employed to coat the microprojections and/or arrays thereof. Preferably, the drug coating formulation includes at least one PTH-based agent, which can be in solution or suspension in the formulation.

The term “controlled release coating formulation”, or “sustained release coating formulation” as used herein, is meant to mean and include a freely flowing composition or mixture that is employed to coat the microprojections and/or arrays thereof on top of at least one drug coating. Preferably, the controlled release coating formulation includes at least one polymer which imparts the controlled release, for example sustained release properties to the coating.

The term “biocompatible coating” and “solid coating”, as used herein, is meant to mean and include a “drug coating formulation” in a substantially solid state.

As indicated above, the present invention generally comprises a delivery system including microprojection member (or system) having a plurality of microprojections (or array thereof) that are adapted to pierce through the stratum corneum into the underlying epidermis layer, or epidermis and dermis layers.

Referring now to FIG. 2, there is shown one embodiment of a microprojection member 30 for use with the present invention. As illustrated in FIG. 2, the microprojection member 30 includes a microprojection array 32 having a plurality of microprojections 34. The microprojections 34 preferably extend at substantially a 90° angle from the sheet, which in the noted embodiment includes openings 38.

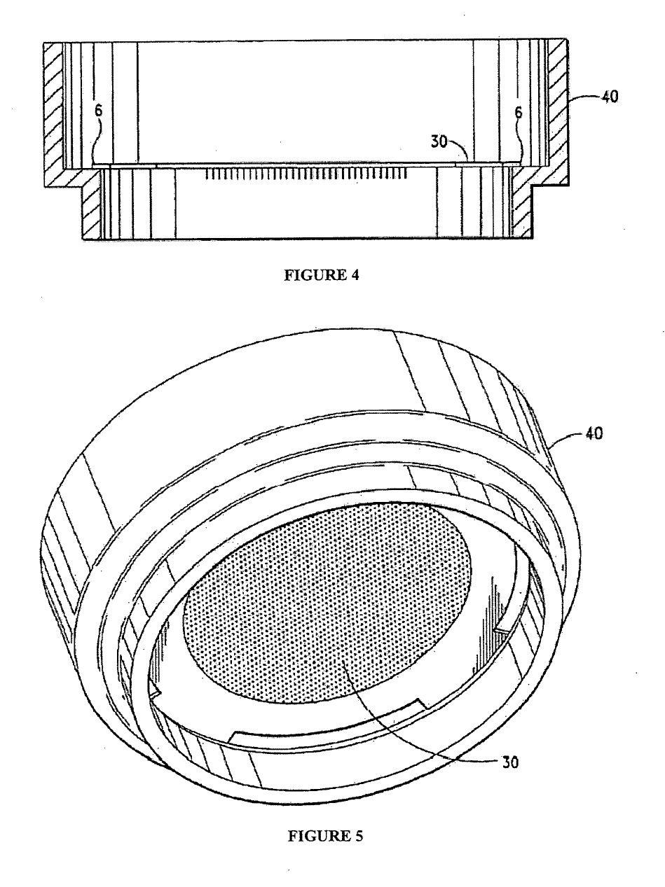

According to the invention, the sheet 36 can be incorporated into a delivery patch, including a backing 40 for the sheet 36, and can additionally include adhesive 16 for adhering the patch to the skin (see FIG. 4). In this embodiment, the microprojections 34 are formed by etching or punching a plurality of microprojections 34 from a thin metal sheet 36 and bending the microprojections 34 out of the plane of the sheet 36.

In one embodiment of the invention, the microprojection member 30 has a microprojection density of at least approximately 10 microprojections/cm², more preferably, in the range of at least approximately 200-2000 microprojections/cm². Preferably, the number of openings per unit area through which the agent passes is at least approximately 10 openings/cm² and less than about 2000 openings/cm².

As indicated, the microprojections 34 preferably have a projection length less than 1000 microns. In one embodiment, the microprojections 34 have a projection length of less than 500 microns, more preferably, less than 250 microns. The microprojections 34 also preferably have a width in the range of approximately 25-500 microns and thickness in the range of approximately 10-100 microns.

In further embodiments of the invention, the biocompatibility of the microprojection member 30 can be improved to minimize or eliminate bleeding and irritation following application to the skin of a subject. Specifically, the microprojections 34 can have a length less than 145 microns, more preferably, in the range of approximately 50-145 microns, and even more preferably, in the range of approximately 70-140 microns. Also, the microprojection member 30 comprises an array preferably having a microprojection density greater than 100 microprojections/cm², and more preferably, in the range of approximately 200-3000 microprojections/cm². Further details regarding microprojection members having improved biocompatibility are found in U.S. Publication No. 20060204562, published Sep. 14, 2006, which is hereby incorporated by reference in its entirety.

The microprojection member 30 can be manufactured from various metals, such as stainless steel, titanium, nickel titanium alloys, or similar biocompatible materials.

According to the invention, the microprojection member 30 can also be constructed out of a non-conductive material, such as a polymeric material. Alternatively, the microprojection member can be coated with a non-conductive material, such as Parylene®, or a hydrophobic material, such as Teflon®, silicon or other low energy material. The noted hydrophobic materials and associated base (e.g., photoresist) layers are set forth in U.S. Application No. 60/484,142, which is incorporated by reference herein in its entirety.

Microprojection members that can be employed with the present invention include, but are not limited to, the members disclosed in U.S. Pat. Nos. 6,083,196, 6,050,988 and 6,091,975, which are incorporated by reference herein in their entirety.

Other microprojection members that can be employed with the present invention include members formed by etching silicon using silicon chip etching techniques or by molding plastic using etched micro-molds, such as the members disclosed U.S. Pat. No. 5,879,326, which is incorporated by reference herein in its entirety.

In certain embodiments of the invention, the microprojections 34 are preferably configured to reduce variability in the applied coating 35. Suitable microprojections generally comprise a location having a maximum width transverse to the longitudinal axis that is located at a position in the range of approximately 25% to 75% of the length of the microprojection from the distal tip. Proximal to the location of maximum width, the width of the microprojection tapers to a minimum width. Further details regarding the noted microprojection configurations are found in U.S. Application Ser. No. 60/649,888, filed Jan. 31, 2005, which is incorporated by reference herein in its entirety.

Referring now to FIG. 3, there is shown a microprojection member 30 having microprojections 34 that include a biocompatible coating 35 that includes a biologically active agent, for example a PTH based agent. According to the invention, the coating 35 can partially or completely cover each microprojection 34. For example, the coating 35 can be in a dry pattern coating on the microprojections 34. The coating 35 can also be applied before or after the microprojections 34 are formed.

According to the invention, the coating 35 can be applied to the microprojections 34 by a variety of known methods. Preferably, the coating is only applied to those portions the microprojection member 30 or microprojections 34 that pierce the skin (e.g., tips 39).

One such coating method comprises dip-coating. Dip-coating can be described as a means to coat the microprojections by partially or totally immersing the microprojections 34 into a coating solution. By use of a partial immersion technique, it is possible to limit the coating 35 to only the tips 39 of the microprojections 34.

A further coating method comprises roller coating, which employs a roller coating mechanism that similarly limits the coating 35 to the tips 39 of the microprojections 34. The roller coating method is disclosed in U.S. application Ser. No. 10/099,604 (Pub. No. 2002/0132054), which is incorporated by reference herein in its entirety. As discussed in detail in the noted application, the disclosed roller coating method provides a smooth coating that is not easily dislodged from the microprojections 34 during skin piercing.

According to the invention, the microprojections 34 can further include means adapted to receive and/or enhance the volume of the coating 35, such as apertures (not shown), grooves (not shown), surface irregularities (not shown) or similar modifications, wherein the means provides increased surface area upon which a greater amount of coating can be deposited.

A further coating method that can be employed within the scope of the present invention comprises spray coating. According to the invention, spray coating can encompass formation of an aerosol suspension of the coating composition. In one embodiment, an aerosol suspension having a droplet size of about 10 to 200 picoliters is sprayed onto the microprojections 10 and then dried.

Pattern coating can also be employed to coat the microprojections 34. The pattern coating can be applied using a dispensing system for positioning the deposited liquid onto the microprojection surface. The quantity of the deposited liquid is preferably in the range of 0.1 to 20 nanoliters/microprojection. Examples of suitable precision-metered liquid dispensers are disclosed in U.S. Pat. Nos. 5,916,524; 5,743,960; 5,741,554; and 5,738,728; which are fully incorporated by reference herein.

Microprojection coating formulations or solutions can also be applied using ink jet technology using known solenoid valve dispensers, optional fluid motive means and positioning means which is generally controlled by use of an electric field. Other liquid dispensing technology from the printing industry or similar liquid dispensing technology known in the art can be used for applying the pattern coating of this invention.

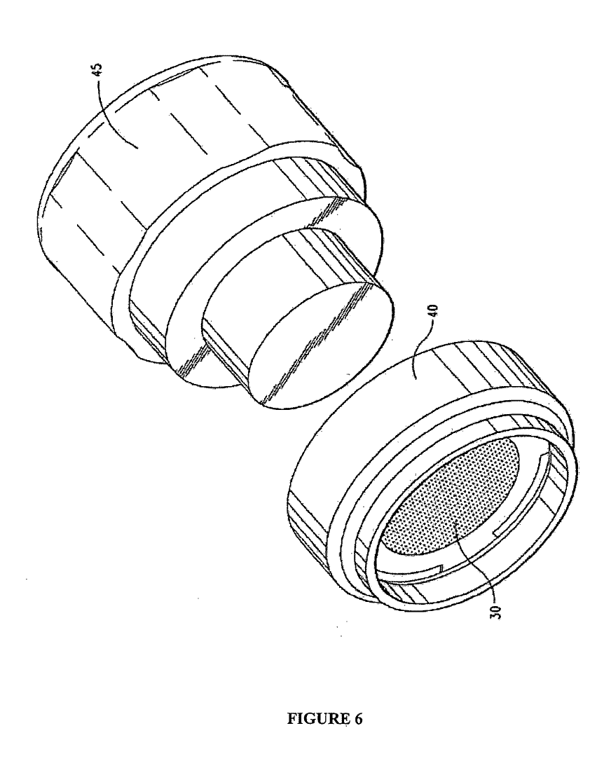

Referring now to FIGS. 5 and 6, for storage and application, the microprojection member 30 is preferably suspended in a retainer ring 40 by adhesive tabs 6, as described in detail in U.S. application Ser. No. 09/976,762 (Pub. No. 2002/0091357), which is incorporated by reference herein in its entirety.

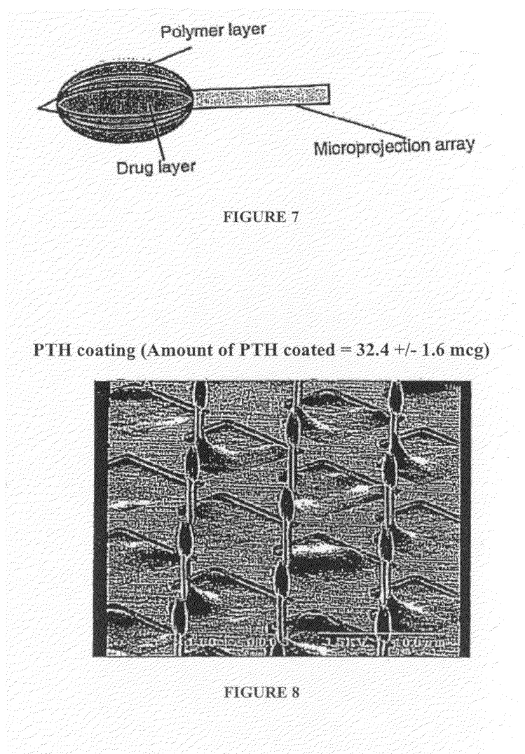

After placement of the microprojection member 30 in the retainer ring 40, the microprojection member 30 is applied to the patient's skin. Preferably, the microprojection member 30 is applied to the patient's skin using an impact applicator 45, such as shown in FIG. 7 and described in Co-Pending U.S. application Ser. No. 09/976,978, which is incorporated by reference herein in its entirety.

As indicated, according to one embodiment of the invention, the drug coating formulations applied to the microprojection member 30 to form solid biocompatible coatings can comprise aqueous and non-aqueous formulations having at least one biologically active agent, for example a PTH based agent. According to the invention, the PTH-based agent can be dissolved within a biocompatible carrier or suspended within the carrier.

In a preferred embodiment, the PTH-based agent is selected from the group consisting of hPTH(1-34), hPTH salts and analogs, teriparatide and related peptides, including, recombinant hPTH(1-34), synthetic hPTH(1-34), PTH(1-34), teriparatide, hPTH(1-34) salts, simple derivatives of hPTH(1-34), such as hPTH(1-34) amide, and closely related molecules, such as hPTH(1-33) or hPTH(1-31) amide, and any other closely related osteogenic peptide. Synthetic hPTH(1-34) is the most preferred PTH-based agent.

Examples of suitable hPTH salts include, without limitation, acetate, propionate, butyrate, pentanoate, hexanoate, heptanoate, levulinate, chloride, bromide, citrate, succinate, maleate, glycolate, gluconate, glucuronate, 3-hydroxyisobutyrate, tricarballylicate, malonate, adipate, citraconate, glutarate, itaconate, mesaconate, citramalate, dimethylolpropinate, tiglicate, glycerate, methacrylate, isocrotonate, □̅hydroxibutyrate, crotonate, angelate, hydracrylate, ascorbate, aspartate, glutamate, 2-hydroxyisobutyrate, lactate, malate, pyruvate, fumarate, tartarate, nitrate, phosphate, benzene, sulfonate, methane sulfonate, sulfate and sulfonate.

Preferably, the biologically active agent, for example a PTH based agent is present in the drug coating formulation at a concentration in the range of approximately 1-30 wt. %.

More preferably, the amount of biologically active agent, for example a PTH based agent contained in the biocompatible coating on the microprojection member is in the range of 1-1000 μg, even more preferably, in the range of 10-100 μg.

Another preferred embodiment is directed to a viscosity-enhancing mixture of counterions, wherein the PTH-based agent has a positive charge at the formulation pH and at least one of the counterions comprises an acid having at least two acidic pKas. The other counterion is an acid with one or more pKas. Examples of suitable acids include, without limitation, hydrochloric acid, hydrobromic acid, nitric acid, sulfuric acid, maleic acid, phosphoric acid, benzene sulfonic acid, methane sulfonic acid, citric acid, succinic acid, glycolic acid, gluconic acid, glucuronic acid, lactic acid, malic acid, pyruvic acid, tartaric acid, tartronic acid, fumaric acid, acetic acid, propionic acid, pentanoic acid, carbonic acid, malonic acid, adipic acid, citraconic acid, levulinic acid, glutaric acid, itaconic acid, meglutol, mesaconic acid, citramalic acid, citric acid, aspartic acid, glutamic acid, tricarballylic acid and ethylenediaminetetraacetic acid.

In another embodiment of the invention, the drug coating formulation includes at least one buffer. Examples of such buffers include, without limitation, ascorbic acid, citric acid, succinic acid, glycolic acid, gluconic acid, glucuronic acid, lactic acid, malic acid, pyruvic acid, tartaric acid, tartronic acid, fumaric acid, maleic acid, phosphoric acid, tricarballylic acid, malonic acid, adipic acid, citraconic acid, glutaratic acid, itaconic acid, mesaconic acid, citramalic acid, dimethylolpropionic acid, tiglic acid, glyceric acid, methacrylic acid, isocrotonic acid, □-hydroxybutyric acid, crotonic acid, angelic acid, hydracrylic acid, aspartic acid, glutamic acid, glycine and mixtures thereof.

In one embodiment of the invention, the drug coating formulation includes at least one antioxidant, which can be sequestering agents, such sodium citrate, citric acid, EDTA (ethylene-dinitrilo-tetraacetic acid) or free radical scavengers such as ascorbic acid, methionine, sodium ascorbate and the like. Presently preferred antioxidants comprise EDTA and methionine.

In the noted embodiments of the invention, the concentration of the antioxidant is in the range of approximately 0.01-20 wt. % of the drug coating formulation. Preferably the antioxidant is in the range of approximately 0.03-10 wt. % of the drug coating formulation.

In one embodiment of the invention, the drug coating formulation includes at least one surfactant, which can be zwitterionic, amphoteric, cationic, anionic, or nonionic, including, without limitation, sodium lauroamphoacetate, sodium dodecyl sulfate (SDS), cetylpyridinium chloride (CPC), dodecyltrimethyl ammonium chloride (TMAC), benzalkonium, chloride, polysorbates, such as Tween 20 and Tween 80, other sorbitan derivatives, such as sorbitan laurate, alkoxylated alcohols, such as laureth-4 and polyoxyethylene castor oil derivatives, such as Cremophor EL®.

In one embodiment of the invention, the concentration of the surfactant is in the range of approximately 0.01-20 wt. % of the drug coating formulation. Preferably the surfactant is in the range of approximately 0.05-1 wt. % of the drug coating formulation.

In a further embodiment of the invention, the drug coating formulation includes at least one polymeric material or polymer that has amphiphilic properties, which can comprise, without limitation, cellulose derivatives, such as hydroxyethylcellulose (HEC), hydroxypropylmethylcellulose (HPMC), hydroxypropycellulose (HPC), methylcellulose (MC), hydroxyethylmethylcellulose (HEMC), or ethylhydroxy-ethylcellulose (EHEC), as well as pluronics.

In one embodiment of the invention, the concentration of the polymer presenting amphiphilic properties in the drug coating formulation is preferably in the range of approximately 0.01-20 wt. %, more preferably, in the range of approximately 0.03-10 wt. % of the drug coating formulation.

In another embodiment, the drug coating formulation includes a hydrophilic polymer selected from the following group: hydroxyethyl starch, carboxymethyl cellulose and salts of, dextran, poly(vinyl alcohol), poly(ethylene oxide), poly(2-hydroxyethylmethacrylate), poly(n-vinyl pyrolidone), polyethylene glycol and mixtures thereof, and like polymers.

In a preferred embodiment, the concentration of the hydrophilic polymer in the drug coating formulation is in the range of approximately 1-30 wt. %, more preferably, in the range of approximately 1-20 wt. % of the drug coating formulation.

In another embodiment of the invention, the drug coating formulation includes a biocompatible carrier, which can comprise, without limitation, human albumin, bioengineered human albumin, polyglutamic acid, polyaspartic acid, polyhistidine, pentosan polysulfate, polyamino acids, sucrose, trehalose, melezitose, raffinose, stachyose, mannitol, and other sugar alcohols.

Preferably, the concentration of the biocompatible carrier in the drug coating formulation is in the range of approximately 2-70 wt. %, more preferably, in the range of approximately 5-50 wt. % of the drug coating formulation.

In another embodiment, the drug coating formulation includes a stabilizing agent, which can comprise, without limitation, a non-reducing sugar, a polysaccharide or a reducing sugar.

Suitable non-reducing sugars for use in the methods and compositions of the invention include, for example, sucrose, trehalose, stachyose, or raffinose.

Suitable polysaccharides for use in the methods and compositions of the invention include, for example, dextran, soluble starch, dextrin, and insulin.

Suitable reducing sugars for use in the methods and compositions of the invention include, for example, monosaccharides such as, for example, apiose, arabinose, lyxose, ribose, xylose, digitoxose, fucose, quercitol, quinovose, rhamnose, allose, altrose, fructose, galactose, glucose, gulose, hamamelose, idose, mannose, tagatose, and the like; and disaccharides such as, for example, primeverose, vicianose, rutinose, scillabiose, cellobiose, gentiobiose, lactose, lactulose, maltose, melibiose, sophorose, and turanose, and the like.

Preferably, the concentration of the stabilizing agent in the drug coating formulation is at ratio of approximately 0.1-2.0:1 with respect to the PTH-based agent, more preferably, approximately 0.25-1.0:1 with respect to the PTH-based agent.

In another embodiment, the drug coating formulation includes a vasoconstrictor, which can comprise, without limitation, amidephrine, cafaminol, cyclopentamine, deoxyepinephrine, epinephrine, felypressin, indanazoline, metizoline, midodrine, naphazoline, nordefrin, octodrine, ornipressin, oxymethazoline, phenylephrine, phenylethanolamine, phenylpropanolamine, propylhexedrine, pseudoephedrine, tetrahydrozoline, tramazoline, tuaminoheptane, tymazoline, vasopressin, xylometazoline and the mixtures thereof. The most preferred vasoconstrictors include epinephrine, naphazoline, tetrahydrozoline indanazoline, metizoline, tramazoline, tymazoline, oxymetazoline and xylometazoline.

As will be appreciated by one having ordinary skill in the art, the addition of a vasoconstrictor to the drug coating formulations and, hence, solid biocompatible coatings of the invention is particularly useful to prevent bleeding that can occur following application of the microprojection member or array and to prolong the pharmacokinetics of the PTH-based agent through reduction of the blood flow at the application site and reduction of the absorption rate from the skin site into the system circulation.

The concentration of the vasoconstrictor, if employed, is preferably in the range of approximately 0.1 wt. % to 10 wt. % of the drug coating formulation.

In another embodiment of the invention, the drug coating formulation includes at least one “pathway patency modulator”, which can comprise, without limitation, osmotic agents (e.g., sodium chloride), zwitterionic compounds (e.g., amino acids), and anti-inflammatory agents, such as betamethasone 21-phosphate disodium salt, triamcinolone acetonide 21-disodium phosphate, hydrocortamate hydrochloride, hydrocortisone 21-phosphate disodium salt, methylprednisolone 21-phosphate disodium salt, methylprednisolone 21-succinate sodium salt, paramethasone disodium phosphate and prednisolone 21-succinate sodium salt, and anticoagulants, such as citric acid, citrate salts (e.g., sodium citrate), dextrin sulfate sodium, aspirin and EDTA.

In yet another embodiment of the invention, the drug coating formulation includes a solubilising/complexing agent, which can comprise Alpha-Cyclodextrin, Beta-Cyclodextrin, Gamma-Cyclodextrin, glucosyl-alpha-Cyclodextrin, maltosyl-alpha-Cyclodextrin, glucosyl-beta-Cyclodextrin, maltosyl-beta-Cyclodextrin, hydroxypropyl beta-Cyclodextrin, 2-hydroxypropyl-beta-Cyclodextrin, 2-hydroxypropyl-gamma-Cyclodextrin, hydroxyethyl-beta-Cyclodextrin, methyl-beta-Cyclodextrin, sulfobutylether-alpha-Cyclodextrin, sulfobutylether-beta-Cyclodextrin, and sulfobutylether-gamma-Cyclodextrin. Most preferred solubilising/complexing agents are beta-Cyclodextrin, hydroxypropyl beta-Cyclodextrin, 2-hydroxypropyl-beta-Cyclodextrin and sulfobutylether7 beta-Cyclodextrin.

The concentration of the solubilising/complexing agent, if employed, is preferably in the range of approximately 1 wt. % to 20 wt. % of the drug coating formulation.

In another embodiment of the invention, the drug coating formulation includes at least one non-aqueous solvent, such as ethanol, isopropanol, methanol, propanol, butanol, propylene glycol, dimethysulfoxide, glycerin, N,N-dimethylformamide and polyethylene glycol 400. Preferably, the non-aqueous solvent is present in the drug coating formulation in the range of approximately 1 wt. % to 50 wt. % of the drug coating formulation.

Other known formulation adjuvants can also be added to the drug coating formulations provided they do not adversely affect the necessary solubility and viscosity characteristics of the drug coating formulation and the physical integrity of the dried coating.

Preferably, the drug coating formulations have a viscosity less than approximately 500 centipoise and greater than 3 centipoise.

In one embodiment of the invention, the thickness of the biocompatible coating is less than 25 microns, more preferably, less than 10 microns, as measured from the microprojection surface.

The desired coating thickness of the drug layer is dependent upon several factors, including the required dosage and, hence, coating thickness necessary to deliver the dosage, the density of the microprojections per unit area of the sheet, the viscosity and concentration of the coating composition and the coating method chosen.

In accordance with one embodiment of the invention, the method for delivering a biologically active agent, for example a PTH based agent contained in the biocompatible coating on the microprojection member includes the following steps: the coated microprojection member is initially applied to the patient's skin via an actuator, wherein the microprojections pierce the stratum corneum. The coated microprojection member is preferably left on the skin for a period lasting from 5 seconds to 24 hours. Following the desired wearing time, the microprojection member is removed.

Preferably, the amount of biologically active agent, for example a PTH based agent contained in the biocompatible coating (i.e., dose) is in the range of approximately 1 μg-1000 μg, more preferably, in the range of approximately 10-200 μg per dosage unit. Even more preferably, the amount of PTH-based agent contained in the biocompatible coating is in the range of approximately 10-100 μg per dosage unit.

In all cases, after a coating has been applied, the coating formulation is dried onto the microprojections 34 by various means. In a preferred embodiment of the invention, the coated microprojection member 30 is dried in ambient room conditions. However, various temperatures and humidity levels can be used to dry the coating formulation onto the microprojections. Additionally, the coated member can be heated, lyophilized, freeze dried or similar techniques used to remove the water from the coating.

It will be appreciated by one having ordinary skill in the art that in order to facilitate drug transport across the skin barrier, the present invention can also be employed in conjunction with a wide variety of iontophoresis or electrotransport systems, as the invention is not limited in any way in this regard. Illustrative electrotransport drug delivery systems are disclosed in U.S. Pat. Nos. 5,147,296, 5,080,646, 5,169,382 and 5,169,383, the disclosures of which are incorporated by reference herein in their entirety.

In many instances, more than one of the noted processes may be occurring simultaneously to different extents. Accordingly, the term “electrotransport” is given herein its broadest possible interpretation, to include the electrically induced or enhanced transport of at least one charged or uncharged agent, or mixtures thereof, regardless of the specific mechanism(s) by which the agent is actually being transported. Additionally, other transport enhancing methods, such as sonophoresis or piezoelectric devices, can be used in conjunction with the invention.

### EXAMPLES

The following examples are given to enable those skilled in the art to more clearly understand and practice the present invention. They should not be considered as limiting the scope of the invention, but merely as being illustrated as representative thereof.

### Example 1

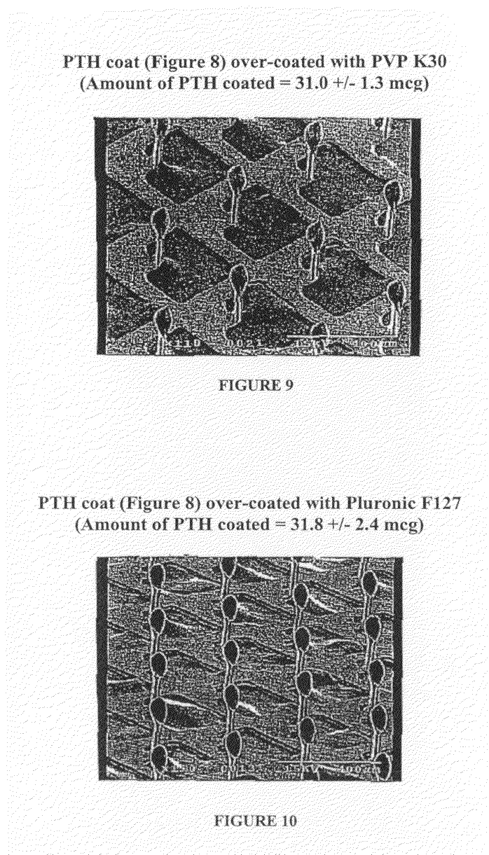

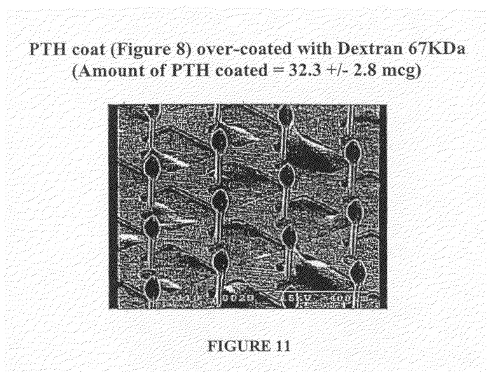

PTH coated microprojection arrays were produced with a polymer layer on top of a drug layer containing PTH. Two reservoirs one containing the PTH drug formulation and one containing a polymer formulation were used in sequence. A microprojection array with the PTH drug formulation coating only is shown in FIG. 8. FIG. 9 shows a microprojection array coated with the PTH formulation with a Pluronic F127 polymer coating on top of the PTH formulation coating. FIG. 10 shows a microprojection array coated with the PTH formulation with a PVP K30 polymer coating on top of the PTH formulation coating. FIG. 11 shows a microprojection array coated with the PTH formulation with a dextran 67 KDa polymer coating on top of the PTH formulation coating. After over coating the PTH content remained unchanged, suggesting that the PTH formulation did not dissolve into the polymer overcoat.

What is claimed is:

1. A transdermal delivery device for delivering a biologically active agent comprising at least one stratum corneum-piercing microprojection, wherein said microprojection has a first coating comprising said biologically active agent and a second coating comprising a polymer, wherein the polymer coating allows controlled release of said biological agent after the transdermal delivery device is applied to the skin of a subject.

2. A transdermal delivery device for delivering a biologically active agent comprising at least one stratum corneum-piercing microprojection, wherein said microprojection has a plurality of coating layers; wherein at least one coating layer comprises said biologically active agent and at least one coating layer comprises a polymer, wherein the polymer coating allows controlled release of said biological agent after the transdermal delivery device is applied to the skin of a subject.

3. The transdermal delivery device of claim 2 wherein coating layers comprising the biologically active agent and coating layers comprising the controlled release polymer are alternately disposed on said microprojection.

4. The device of claim 1 wherein the polymer is a hydrophilic polymer or a hydrophobic PLGA copolymer.

5. The device of claim 1 wherein the polymer layer has a thickness selected to provide a predetermined sustained release profile for the biologically active agent.

6. The device of claim 1 wherein the polymer layer has a copolymer molar mass, a copolymer architecture, a water hydration rate, and/or or layer thickness selected to provide a predetermined sustained release profile for the biologically active agent.

7. The device of claim 1 wherein the controlled release profile comprises a shorter tmax and a rapid concentration drop off.

8. The device of claim 1 wherein the controlled release comprises a reduced Cmax and an extended drop-off tail.

9. The device of claim 1 wherein the polymer layer encapsulates the biologically active agent and slows down release of the biologically active agent.

10. The device of claim 1 wherein the biologically active agent is selected from the group consisting of growth hormone release hormone (GHRH), growth hormone release factor (GHRF), insulin, insultropin, calcitonin, octreotide, endorphin, TRN, NT-36 (chemical name: N-[[(s)-4-oxo-2-azetidinyl] carbonyl]-L-histidyl-L-prolinamide), liprecin, pituitary hormones (e.g., HGH, HMG, desmopressin acetate, etc), follicle luteoids, aANF, growth factors such as growth factor releasing factor (GFRF), bMSH, GH, somatostatin, bradykinin, somatotropin, platelet-derived growth factor releasing factor, asparaginase, bleomycin sulfate, chymopapain, cholecystokinin, chorionic gonadotropin, erythropoietin, epoprostenol (platelet aggregation inhibitor), gluagon, HCG, hirulog, hyaluronidase, interferon alpha, interferon beta, interferon gamma, interleukins, interleukin-10 (IL-10), erythropoietin (EPO), granulocyte macrophage colony stimulating factor (GM-CSF), granulocyte colony stimulating factor (G-CSF), glucagon, leutinizing hormone releasing hormone (LHRH), LHRH analogs (such as goserelin, leuprolide, buserelin, triptorelin, gonadorelin, and napfarelin, menotropins (urofollitropin (FSH) and LH)), oxytocin, streptokinase, tissue plasminogen activator, urokinase, vasopressin, deamino [Val4, D-Arg8] arginine vasopressin, desmopressin, corticotropin (ACTH), ACTH analogs such as ACTH (1-24), ANP, ANP clearance inhibitors, angiotensin II antagonists, antidiuretic hormone agonists, bradykinn antagonists, ceredase, CSI's, calcitonin gene related peptide (CGRP), enkephalins, FAB fragments, IgE peptide suppressors, IGF-1, neurotrophic factors, colony stimulating factors, parathyroid hormone and agonists, parathyroid hormone antagonists, parathyroid hormone (PTH), PTH analogs such as PTH (1-34), prostaglandin antagonists, pentigetide, protein C, protein S, renin inhibitors, thymosin alpha-1, thrombolytics, TNF, vasopressin antagonists analogs, alpha-1 antitypsin (recombinant), and TGF-beta.

11. The device of claim 1, wherein the biologically active agent comprises hPTH.

12. The device of claim 1, wherein the biologically active agent comprises hPTH(1-34) and analogs.

13. The device of claim 1, wherein said biologically active agent is growth hormone releasing factor (GRF), parathyroid hormone (PTH), parathyroid hormone related protein (PTHrp), calcitonin, or a soluble, biologically active analog of GRF, PTH, PTHrp, or calcitonin.

13. The device of claim 1, wherein said microprojection has a length of less than about 500 micrometers and a thickness of less than about 25 micrometers.

14. The device of claim 1, wherein said stratum corneum-piercing microprojection is formed by etching said microprotrusion from a thin sheet and folding said microprojection out of a plane of the sheet.

15. A method for forming a device for transdermally delivering a biologically active agent comprising the steps of: forming at least one stratum corneum-piercing microprojection in a thin sheet of material; applying a first coating comprising said biologically active agent and a second coating comprising a polymer, wherein the polymer coating allows controlled release of said biological agent after the transdermal delivery device is applied to the skin of a subject.

16. The method of claim 15, further comprising the step of bending said microprojection out of a plane formed by said thin sheet after applying said first and second coatings.

17. The method of claim 15, wherein the step of forming said microprojection is selected from the group consisting of etching and punching.

18. A transdermal delivery device for delivering a biologically active agent comprising a microprojection array of a plurality of stratum corneum-piercing microprojections, wherein at least a portion of each of said microprojections has a first coating comprising said biologically active agent and a second coating comprising a polymer, wherein the polymer coating allows controlled release of said biological agent after the transdermal delivery device is applied to the skin of a subject.

19. The device of claim 18, wherein said microprojection array has a density of at least about 10 microprojections/cm2.

20. The device of claim 18, wherein said microprojection array has a density of about 200-2000 microprojections/cm2.
